(19) United States
(12) Patent Application Publication
(10) Pub. No.: US 2026/0360478 A1
(43) Pub. Date: Nov. 12, 2026

(54) METHOD FOR PREDICTING NATURAL ABUNDANCE OF NITROGEN ISOTOPES IN AMMONIA VOLATILIZATION FROM CROPLAND BASED ON ENVIRONMENTAL AND SOIL FACTORS

(71) Applicant: Institute of Soil Science, Chinese Academy of Sciences, Nanjing (CN)

(72) Inventors: Chaopu Ti, Nanjing (CN); Lingyun Peng, Nanjing (CN); Xiaoyuan Yan, Nanjing (CN)

(21) Appl. No.: 19/637,479

(22) Filed: Apr. 2, 2026

(30) Foreign Application Priority Data
May 6, 2025 (CN) 202510570710.6

(51) Int. Cl. G16C 20/30; G01N 33/24; G16C 20/70

(52) U.S. Cl. CPC G16C 20/30; G01N 33/245; G16C 20/70

(57) ABSTRACT

The present disclosure relates to the interdisciplinary technical field of agro-environmental science and isotope ecology, and provides a method for predicting natural abundance of nitrogen isotopes during ammonia volatilization based on environmental and soil factors. The method includes candidate indicator dataset collection, determination of an ammonia volatilization nitrogen natural abundance dataset, model construction, model training, calculation of a candidate indicator SHAP dataset, indicator filtering, secondary training, error ratio calculation, output of the prediction model and prediction thereof. The present disclosure optimizes the candidate indicator set through indicator SHAP data, achieving a reduction in model parameters and improving the prediction efficiency of the model. Through the Keras Sequential improved model, the integration of data correlations and different training strategies for various types of indicators are achieved, addressing an overfitting risk during model training and enhancing the prediction accuracy of the model for the natural abundance.

### CROSS-REFERENCE TO RELATED APPLICATION

This application claims priority of Chinese Patent Application No. 202510570710.6, filed on May 6, 2025, the entire contents of which are incorporated herein by reference.

### TECHNICAL FIELD

The present disclosure relates to the cross-disciplinary technical field of agricultural environmental science and isotope ecology, particularly to a method for predicting natural abundance of nitrogen isotopes in ammonia volatilization based on environmental and soil factors.

### BACKGROUND

As the primary alkaline gas in the atmosphere and a key component of reactive nitrogen, ammonia plays a crucial role in atmospheric chemical processes and the soil nitrogen cycle. However, the release of large amounts of ammonia into the environment has many negative impacts on human health and ecosystem functions. For example, ammonia in the air readily reacts with acidic substances such as sulfur dioxide and nitrogen oxides. These reactions form important precursors of PM_{2.5}, which exacerbates air pollution and poses serious risks to human health. Besides, ammonia enters terrestrial and marine ecosystems through atmospheric nitrogen deposition, which may lead to soil acidification, water eutrophication, and reduced biodiversity. Therefore, identifying and quantifying the contribution of cropland systems to atmospheric ammonia is the foundation for effective emission reduction. The natural abundance of nitrogen isotope is a valuable tool for identifying and quantifying the sources of atmospheric ammonia, and can reflect the sources, transformations, and fate of nitrogen within ecosystems.

However, conventional nitrogen isotope analysis methods primarily rely on laboratory isotope mass spectrometry, which are not only time-consuming and labor-intensive but also difficult to monitor dynamic changes during ammonia volatilization for croplands. Currently, calculation methods for natural abundance of nitrogen isotopes during ammonia volatilization for cropland are still inadequate, and an efficient algorithm that comprehensively accounts for both environmental and soil factors is lacking.

### SUMMARY

To overcome the deficiencies of existing technologies, the present disclosure aims to provide a method for predicting the natural abundance of nitrogen isotopes based on environmental and soil factors. This method effectively resolves the low efficiency of conventional methods used to analyze nitrogen isotopes during ammonia volatilization.

To achieve this objective, the present disclosure provides the following solutions.

A method for predicting the natural abundance of nitrogen isotopes during ammonia volatilization based on environmental and soil factors includes the following steps:
Determining a candidate indicator set and, based on this set, performing data collection and preprocessing within the target study area to generate a candidate indicator dataset;
Collecting natural abundance values of nitrogen isotopes during ammonia volatilization for cropland within the target study area using isotope mass spectrometry analysis to obtain a natural abundance dataset;
Constructing a Keras Sequential model by inserting a feature-level self-attention module between an input layer and a first hidden layer, adding a time decay factor before a Dropout layer, replacing the original bias in an activation function of hidden layers with a fixed bias and a variable bias based on fixed and variable quantities in the candidate indicator set, and determining activation of the fixed bias and the variable bias through an indicator function to obtain an improved Keras Sequential model;
Constructing a loss function, and iteratively training the improved Keras Sequential model using the candidate indicator dataset and the natural abundance dataset based on the loss function and an early stopping method to obtain an original natural abundance prediction model;
Calculating the SHAP value of each candidate indicator in the original natural abundance prediction model using a DeepSHAP interpreter to obtain a candidate indicator SHAP dataset;
Filtering the candidate indicator SHAP dataset using a preset SHAP threshold to obtain an updated candidate indicator set;
Training the improved Keras Sequential model using the candidate indicator dataset and the data corresponding to the candidate indicator set in the natural abundance dataset to obtain the indicator-optimized natural abundance prediction model;
Calculating the error degree of the indicator-optimized natural abundance prediction model relative to the original natural abundance prediction model using a preset error function to obtain an error ratio;
When the error ratio is less than a preset error threshold, determining the indicator-optimized natural abundance prediction model as a nitrogen isotope natural abundance prediction model; and
Performing prediction for the target cropland area using the nitrogen isotope natural abundance prediction model to obtain a prediction result.

Preferably, the iterative training process of the Keras Sequential model includes:
Converting the candidate indicator dataset into tensor form to obtain input data;
Performing linear transformation, attention weight calculation, weighted fusion, residual connection, and normalization on the input data using the feature-level self-attention module to obtain an attention output;
Inputting the attention output into the hidden layer for abstraction processing to obtain a feature tensor;
Dynamically retaining and randomly masking the feature tensor using the Dropout layer to obtain a sparsified tensor;
Calculating the sparsified tensor sequentially using the remaining hidden layers and the Dropout layer of the Keras Sequential model to obtain a feature extraction result; and
Inputting the feature extraction result into a fully connected layer to map nitrogen isotope abundance values during ammonia volatilization, thereby generating an iterative prediction result.

Preferably, the activation function is expressed as follows:

f(xᵢ)=ReLU(wᵢxᵢ+b_{fixed}·I(Tₓ_{ᵢ}∈T₀)+bᵥₐᵣ·I(Tₓ_{ᵢ}∈T₁));
wherein f(xᵢ) denotes the output of the activation function; ReLU(·) denotes the activation function; wᵢ represents a weight parameter; xᵢ denotes an eigenvalue; b_{fixed} represents a fixed bias; bᵥₐᵣ represents a variable bias; I(·) represents an indicator function; Tₓ_{ᵢ} represents an indicator type corresponding to the eigenvalue; T₀ represents a fixed indicator; and T₁ represents a variable indicator.

Preferably, the error function is expressed as follows:

$$E = \sum \frac{\vert \text{?} - {y}_{n} \vert}{\vert \text{?} - {y}_{n} \vert} ;$$

wherein E denotes an error ratio; [symbol] denotes a prediction result of the original natural abundance prediction model; [symbol] denotes a prediction result of the indicator-optimized natural abundance prediction model; and yₙ denotes a measured value of the natural abundance.

Preferably, the determined candidate indicator set includes: air temperature, atmospheric humidity, soil pH, soil ammonium-nitrogen concentration, soil nitrate-nitrogen concentration, soil type, land-use type, fertilization type, nitrogen fertilizer application rate, temperature change rate, humidity change rate, ammonium-nitrogen concentration change rate, nitrate-nitrogen concentration change rate, precipitation, wind speed, light intensity, soil moisture, and soil electrical conductivity.

Preferably, the SHAP threshold ranges from 0.1 to 0.2.

Preferably, the data preprocessing includes outlier removal, linear interpolation, mode imputation, and data normalization.

Preferably, the feature-level self-attention module is expressed as follows:

$$Output = LayerNorm ( X + Softmax ( \frac{{XW}_{Q} \cdot {( {XW}_{K} )}^{T}}{\sqrt{d}} ) \cdot ( {XW}_{V} ) \cdot {W}_{O} ) ;$$

wherein Output is an attention output; LayerNorm(·) represents a layer normalization operation; Softmax(·) represents a normalized exponential function; X is an input data matrix; W_{Q}, W_{K}, and W_{V} and are a query matrix, a key matrix, and a value matrix, respectively; W_{O} is an output projection weight matrix; and d is a scaling factor.

Preferably, the Dropout layer is expressed as follows:

$$Dropout ( {X}_{t} , t ) = \{ \begin{matrix}\frac{{X}_{t} ⊙ {M}_{t}}{p ( t )} \\ {X}_{t}\end{matrix} ;$$

wherein Dropout(Xₜ, t) is an output of the Dropout layer; Xₜ is an input tensor; Mₜ is a random mask matrix; p(t) is a retention probability; and ⊙ represents an element-wise multiplication operation.

Preferably, the collection equipment for the candidate indicator set includes a glass electrode pH sensor, a digital ammonia nitrogen and nitrate nitrogen measurement sensor, a resistive soil moisture sensor, and a conductivity sensor.

The present disclosure discloses the following technical effects:

The present disclosure provides a method for predicting natural abundance of nitrogen isotopes during ammonia volatilization based on environmental and soil factors. By optimizing the candidate indicator set using the SHAP data, the present disclosure addresses the issues of excessive size and low efficiency in conventional pre-training models, thereby reducing model parameters. The improved Keras Sequential model overcomes the limitations of the existing model, which lacks synchronized training strategies for different metrics and suffers from insufficient data correlation processing. This enables the integration of inter-data correlations and the implementation of distinct training strategies for different types of metrics.

### BRIEF DESCRIPTION OF DRAWINGS

To more clearly illustrate the technical solutions in the embodiments of the present disclosure or the existing technologies, the accompanying drawings required for describing the embodiments will be briefly introduced below. The accompanying drawings described below illustrate only certain embodiments of the present disclosure. For those skilled in the art, other accompanying drawings may be derived from these drawings without creative effort.

FIG. 1 is a schematic diagram of the prediction process for natural abundance of nitrogen isotopes during ammonia volatilization based on environmental and soil factors provided by an embodiment of the present disclosure.

FIG. 2 is a schematic diagram of the training process of the improved Keras Sequential model provided by an embodiment of the present disclosure.

FIG. 3 is a scatter distribution graph of predicted values versus measured values of a pre-trained model provided by an embodiment of the present disclosure.

FIG. 4 is a scatter distribution plot of predicted values versus measured values of a multivariate linear model according to an embodiment of the present disclosure.

FIG. 5 is a schematic diagram of importance evaluation provided by an embodiment of the present disclosure.

### DETAILED DESCRIPTION OF EMBODIMENTS

The technical solutions in the embodiments of the present disclosure will be clearly and completely described below with reference to the accompanying drawings. The described embodiments represent only a part of the embodiments of the present disclosure rather than all embodiments. Based on the embodiments of the present disclosure, all other embodiments obtained by those skilled in the art without creative efforts shall fall within the scope of protection of the present disclosure.

The objective of the present disclosure is to provide a method for predicting natural abundance of nitrogen isotopes during ammonia volatilization based on environmental and soil factors, addressing the issue of excessively low efficiency in conventional methods for analyzing nitrogen isotopes during ammonia volatilization.

To make the above objectives, features, and advantages of the present disclosure more comprehensible, the present disclosure is described in further detail below with reference to the accompanying drawings and specific embodiments.

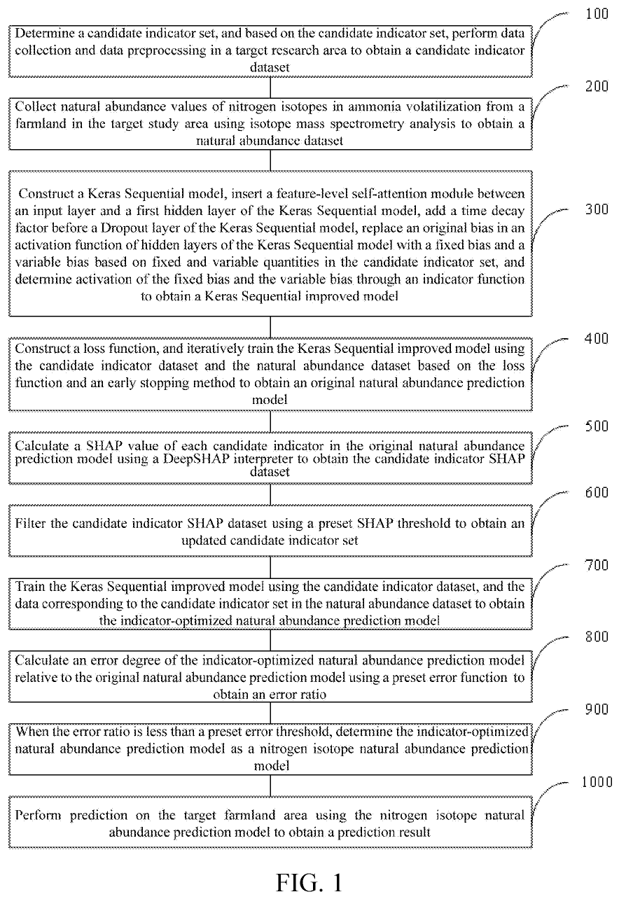

FIG. 1 is a schematic diagram of the prediction process for natural abundance of nitrogen isotopes during ammonia volatilization based on environmental and soil factors provided by an embodiment of the present disclosure. As shown in FIG. 1, the present disclosure provides a method for predicting natural abundance of nitrogen isotopes during ammonia volatilization based on environmental and soil factors, which includes the following steps.

In step 100, a candidate indicator set is determined, and based on the candidate indicator set, data collection and data preprocessing are performed in a target study area to obtain a candidate indicator dataset.

In step 200, natural abundance values of nitrogen isotopes during ammonia volatilization are collected from cropland within the target study area using isotope mass spectrometry analysis to obtain a natural abundance dataset.

In step 300, a Keras Sequential model is constructed, a feature-level self-attention module is inserted between an input layer and a first hidden layer, a time decay factor is added before a Dropout layer, an original bias in an activation function of hidden layers is replaced with a fixed bias and a variable bias based on fixed and variable quantities in the candidate indicator set, and activation of the fixed bias and the variable bias is determined through an indicator function to obtain the improved Keras Sequential model.

In step 400, a loss function is constructed, and the improved Keras Sequential model is iteratively trained using the candidate indicator dataset and the natural abundance dataset based on the loss function and an early stopping method to obtain an original natural abundance prediction model.

In step 500, a SHAP value of each candidate indicator in the original natural abundance prediction model is calculated using a DeepSHAP interpreter to obtain the candidate indicator SHAP dataset.

In step 600, the candidate indicator SHAP dataset is filtered using a preset SHAP threshold to obtain an updated candidate indicator set.

In step 700, the improved Keras Sequential model is trained using the candidate indicator dataset, the data corresponding to the candidate indicator set in the natural abundance dataset to obtain the indicator-optimized natural abundance prediction model.

In step 800, an error degree of the indicator-optimized natural abundance prediction model relative to the original natural abundance prediction model is calculated using a preset error function to obtain an error ratio.

In step 900, when the error ratio is less than a preset error threshold, the indicator-optimized natural abundance prediction model is determined as a nitrogen isotope natural abundance prediction model.

In step 1000, prediction is performed on the target farmland area using the nitrogen isotope natural abundance prediction model to obtain a prediction result.

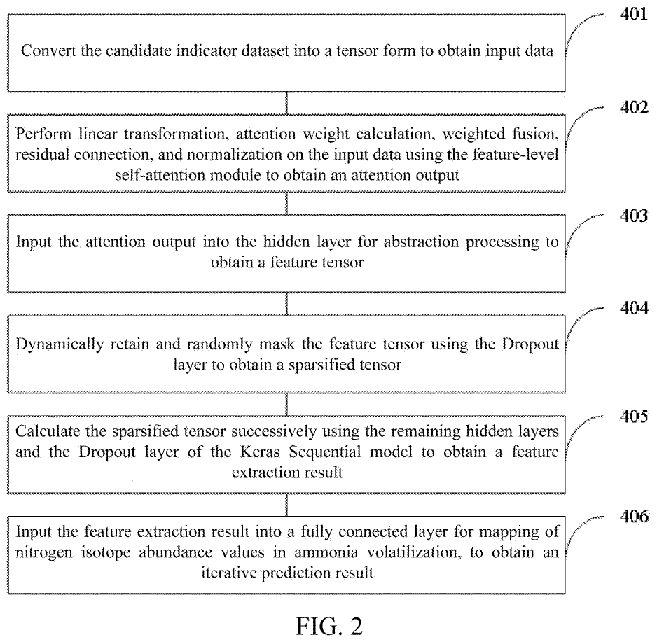

Referring to FIG. 2, the iterative training process of the Keras Sequential model includes the following steps.

In step 401, the candidate indicator dataset is converted into a tensor form to obtain input data.

In step 402, linear transformation, attention weight calculation, weighted fusion, residual connection, and normalization are performed on the input data using the feature-level self-attention module to obtain an attention output.

In step 403, the attention output is input into the hidden layer for abstraction processing to obtain a feature tensor.

In step 404, the feature tensor is dynamically retained and randomly masked using the Dropout layer to obtain a sparsified tensor.

In step 405, the sparsified tensor is calculated successively using the remaining hidden layers and the Dropout layer of the Keras Sequential model to obtain a feature extraction result.

In step 406, the feature extraction result is input into a fully connected layer for mapping of nitrogen isotope abundance values during ammonia volatilization, to obtain an iterative prediction result.

Further, the activation function is expressed as follows:

f(xᵢ)=ReLU(wᵢxᵢ+b_{fixed}·I(Tₓ_{ᵢ}∈T₀)+bᵥₐᵣ·I(Tₓ_{ᵢ}∈T₁));
wherein f(xᵢ) denotes the output of the activation function; ReLU(·) denotes the activation function; wᵢ represents a weight parameter; xᵢ denotes an eigenvalue; b_{fixed} represents a fixed bias; bᵥₐᵣ represents a variable bias; I(·) represents an indicator function; Tₓ_{ᵢ} represents an indicator type corresponding to the eigenvalue; T₀ represents a fixed indicator; and T₁ represents a variable indicator.

Specifically, the error function is expressed as follows:

$$E = \sum \frac{\vert \text{?} - {y}_{n} \vert}{\vert \text{?} - {y}_{n} \vert} ;$$

wherein E denotes an error ratio; [symbol] denotes a prediction result of the original natural abundance prediction model; [symbol] denotes a prediction result of the indicator-optimized natural abundance prediction model; and yₙ denotes a measured value of the natural abundance.

Optionally, the determined candidate indicator set includes air temperature, atmospheric humidity, soil pH, soil ammonium-nitrogen concentration, soil nitrate-nitrogen concentration, soil type, land-use type, fertilization type, nitrogen fertilizer application rate, temperature change rate, humidity change rate, ammonium-nitrogen concentration change rate, nitrate-nitrogen concentration change rate, precipitation, wind speed, light intensity, soil moisture, and soil electrical conductivity.

Specifically, the SHAP threshold is ranged from 0.1 to 0.2.

Preferably, the data preprocessing includes: outlier removal, linear interpolation, mode imputation, and data normalization.

Further, the feature-level self-attention module is expressed as follows:

$$Output = LayerNorm ( X + Softmax ( \frac{{XW}_{Q} \cdot {( {XW}_{K} )}^{T}}{\sqrt{d}} ) \cdot ( {XW}_{V} ) \cdot {W}_{O} ) ;$$

wherein Output is an attention output; LayerNorm(·) represents a layer normalization operation; Softmax(·) represents a normalized exponential function; X is an input data matrix; W_{Q}, W_{K}, and W_{V} and are a query matrix, a key matrix, and a value matrix, respectively; W_{O} is an output projection weight matrix; and d is a scaling factor.

Specifically, the Dropout layer is expressed as follows:

$$Dropout ( {X}_{t} , t ) = \{ \begin{matrix}\frac{{X}_{t} ⊙ {M}_{t}}{p ( t )} \\ {X}_{t}\end{matrix} ;$$

wherein Dropout(Xₜ, t) is an output of the Dropout layer; Xₜ is an input tensor; Mₜ is a random mask matrix; p(t) is a retention probability; and ⊙ represents an element-wise multiplication operation.

Preferably, the collection equipment for the candidate indicator set includes a glass electrode pH sensor, a digital ammonia-nitrogen and nitrate-nitrogen measurement sensor, a resistive soil moisture sensor, and a conductivity sensor.

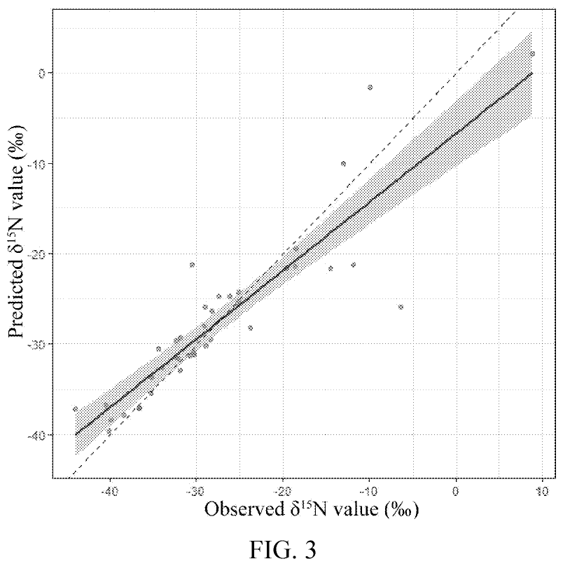

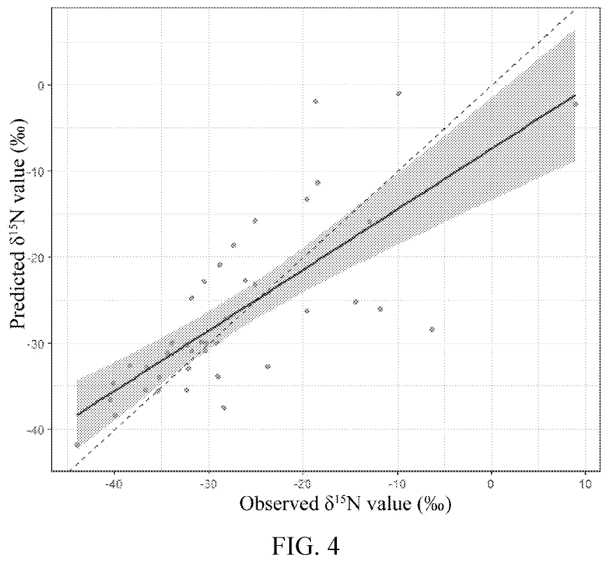

Further, FIG. 3 is a scatter distribution graph of predicted values versus measured values for a pre-trained model, and FIG. 4 is a scatter distribution graph of predicted values versus measured values for a multiple linear model. By comparison, it can be determined that the pre-trained model in this embodiment is more accurate than the multiple linear model in predicting the natural abundance of nitrogen isotopes during ammonia volatilization for cropland.

Specifically, data collection and preprocessing include the natural abundance values of nitrogen isotopes during ammonia volatilization for cropland in the target area are obtained, and corresponding environmental factor datasets and soil factor datasets are simultaneously collected. The environmental factors mentioned include temperature, humidity, etc., while the soil factors include soil pH, ammonium-nitrogen concentration, nitrate-nitrogen concentration, soil type, etc.; missing value imputation, standardization, or normalization are performed on the data, and the training and test set are constructed. The predictive performance of the two models is validated using the test set, and the model accuracy is compared using the coefficient of determination (R²), root mean square error (RMSE), and mean absolute error (MAE).

Further, data preprocessing includes performing outlier removal using a box plot method or a 3σ principle, wherein data points exceeding 1.5 times the interquartile range or falling outside the mean±3 times the standard deviation are defined as outliers. Samples with missing values exceeding the threshold (≥20%) are excluded, and the remaining missing values are imputed using the Multiple Imputation by Chained Equations (MICE) method, with the number of imputation iterations set to 10 and the convergence tolerance set to 0.001. For continuous variables, Z-score normalization is applied using the following formula:

$$z = \frac{x - μ}{σ} ;$$

wherein z is a standard output, x is data to be processed, μ is a mean, and σ is a standard deviation; one-hot encoding is performed on categorical variables.

Preferably, a time decay function is used to dynamically adjust the neuron retention probability, alleviating the issue of noise accumulation in time series data. A calculation formula thereof is as follows:

p(t)=0.8·e^{-0.01t};
wherein t represents a time step.

Optionally, this embodiment directly obtain data such as temperature and humidity from weather report websites while ensuring that the temporal resolution and spatial coverage match the requirements of cropland prediction. Static indicators such as soil type need to be collected once at the beginning of the prediction period, while dynamic indicators such as ammonium-nitrogen concentration require high-frequency predictions (once per hour).

Optionally, tensorization transformation includes transforming the preprocessed structured data into a three-dimensional tensor (number of samples×time steps×number of features). For example, hourly data collected over five consecutive days form a 120×18 tensor slice. The time dimension generates sequence samples through a sliding window, with the window length set to 24 (representing a daily cycle) and the step size set to 6, enabling overlapping sampling to enhance data utilization.

Specifically, in the linear projection layer, the input tensor is first passed through three independent fully connected layers to generate query (Query), key (Key), and value (Value) matrices, respectively. The projection dimension is set to one-fourth of the original feature count, achieving a balance between computational efficiency and information retention. For example, the weight matrices W_Q, W_K, and W_V for projecting 18-dimensional features into a 4-dimensional space have dimensions of 18×4.

Further, the attention weight calculation obtains a similarity matrix between features through matrix multiplication. Specifically, the query matrix is multiplied by the transposed key matrix, with the scaling factor d set to the square root of the projection dimension. The obtained similarity matrix is normalized to generate an attention weight matrix. Each element of this matrix reflects an association strength between two features in the current context.

Further, feature fusion and residual connection include the following steps. The value matrix is weighted and summed using attention weights to obtain a context-aware feature representation. The output projection layer maps the fused features back to the original dimensions. To prevent the information loss, the original input is added to the attention output, and then layer normalization is applied to stabilize gradient propagation. This process enables the model to autonomously identify key feature combinations, such as a synergistic effect between the pH value of soil and the ammonium-nitrogen concentration.

Specifically, during a model initialization phase, a type mapping table is established based on the properties of the characteristics. Static indicators (such as soil type) are labeled as T₀, while dynamic indicators (such as temperature change rate) are labeled as T₁. Each input feature carries a corresponding type label into the network.

Preferably, the hidden layer activation function has two sets of bias parameters built-in: b_{fixed} (fixed bias) and bᵥₐᵣ (variable bias). During forward propagation, the corresponding bias term is enabled based on the feature type label. The indicator function I(·) essentially serves as a conditional gate, b_{fixed} participating in the calculation when the feature belongs to T₀. When belonging to T₁, bᵥₐᵣ takes effect. This mechanism enables the network to establish stable representations for static features while maintaining flexible responses to dynamic features.

Optionally, the fixed bias uses a smaller learning rate (one-tenth of the model learning rate) to prevent over-adjustment of parameters related to static features. A change bias using adaptive learning rate optimizers (such as Nadam) to quickly capture the dynamic patterns of metrics. Gradients of the two types of bias are computed separately during backpropagation to ensure training stability.

Preferably, the time decay Dropout layer is designed with a retention probability decay curve. The initial retention probability is set to 0.8, with a decay coefficient of 0.01. Here, t represents the number of training epochs (time steps). After each completed epoch, the global retention probability decreases by approximately 1%. This design initially allows a large number of neurons to participate in learning, and gradually becomes sparse in later stages to suppress overfitting.

Further, dynamic mask generation involves creating a new random mask matrix Mₜ for each training iteration, wherein the elements take values of 0 or 1, with the probability of occurrence determined by the current p(t). In the first 100 epochs, p(t) gradually decreases from 0.8 to 0.29, maintaining strong learning capability in the network. After 300 epochs, p(t) approaches 0.04, forming a highly sparse connection pattern.

Specifically, during the inference phase, the Dropout layer is turned off, but the average activation strength of neurons in each layer from the training phase is retained. By recording typical mask patterns at the end of training, weights are scaled and compensated during inference to maintain consistency in output magnitude.

Preferably, the loss function adopts the Huber loss function, which adaptively switches between the mean squared error (MSE) and the mean absolute error (MAE), with a threshold set to 1.0. When the prediction deviation is less than the threshold, the MSE is used to promote convergence; when it exceeds the threshold, it switches to the MAE to enhance robustness.

Optionally, the smoothed loss value is monitored on a validation set: a moving average of the loss over the last 10 epochs is calculated, and early stopping is triggered when the average loss fails to decrease for three consecutive times. To prevent misjudgment due to occasional fluctuations, a patience coefficient of 15 epochs is set, allowing the loss to fluctuate within the threshold range.

Specifically, the Nadam optimizer (Adam+NAG momentum) is used with an initial learning rate 3e⁻⁴, combined with a triangular cyclic learning rate schedule. Every 5 epochs, it varies cyclically between 1e⁻⁴ and 3e⁻⁴, facilitating escape from local optima. The batch size is set to 64 to balance memory efficiency with gradient estimation stability.

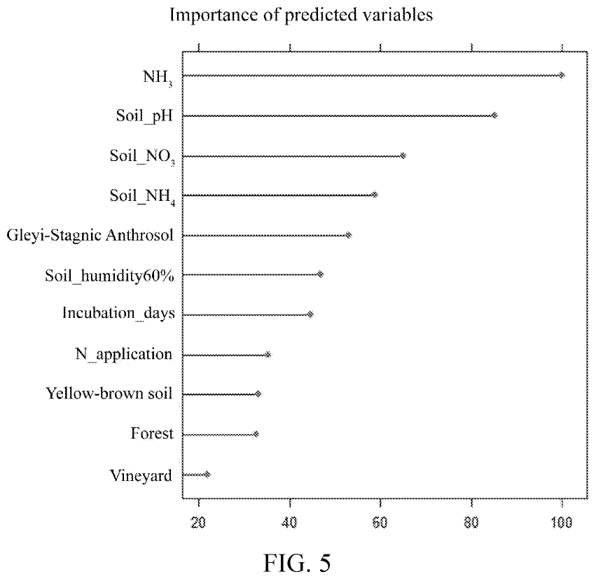

Further, after the initial training is completed, perturbation analysis is performed on validation set samples. Each feature is subjected to a ±10% value perturbation in sequence, and the magnitude of change in the model output is recorded. SHAP values are calculated through Monte Carlo sampling to quantify the marginal contribution of each feature to the prediction results. The SHAP values are mapped to a percentage scale, as shown in FIG. 5, wherein NH₃ represents the ammonia concentration, soil_pH represents the pH value of soil, and soil_NH₄ represents the soil ammonium nitrogen concentration. Soil_NO₃ indicates the nitrate nitrogen concentration of soil, Gleyi-Stagnic Anthrosol refers to Gleyi-Stagnic Anthrosol, soil_humidity represents the soil moisture, and incubation_days represents the measurement days. N_application represents the nitrogen fertilizer application level; Yellow-brown soil represents yellow-brown earth; Forest represents a forest; Vineyard represents an agricultural land used for grape cultivation.

The environmental factors include air temperature, humidity, etc., and the soil factors include soil pH, ammonium-nitrogen concentration, nitrate-nitrogen concentration, soil type, etc.; and missing value imputation, standardization, or normalization operations are performed on the data.

Further, the threshold screening strategy sets the absolute SHAP value threshold to 60% of the maximum value (adjustable based on data scale), retaining the top 60% of features by contribution. Bundled screening is performed on strongly correlated feature groups (such as temperature and rate of temperature change) to avoid mistakenly deleting associated features. The selected feature set must satisfy the following conditions simultaneously. The SHAP value of a single feature is greater than the threshold, and at least one representative item is retained within the feature group.

Optionally, three test areas with different climate types outside the target region are selected, and fine-tuning is performed using transfer learning. The weights of the first 3 layers are frozen, and only the final fully connected layer is trained. The fine-tuning period is limited to 20 epochs to validate the environmental adaptability of the model.

Preferably, online prediction deployment includes converting the final model into TensorRT format and deploying it on an edge computing device. A two-level cache mechanism is designed, wherein real-time data is first stored in a circular buffer, and model inference is triggered once every full day of data. The output results are uploaded to the cloud platform via the LoRa wireless module, enabling unattended prediction in the field.

Further, the training process is monitored visually. The first-layer self-attention matrix and plot feature correlation heatmaps are periodically exported. By analyzing the evolution trend of attention weights, it can be verified whether the model learns the expected physicochemical relationships. The activation value distribution of each hidden layer is recorded to monitor for the presence of gradient vanishing or explosion. Ideally, the values after ReLU activation should exhibit a right-skewed distribution. Time series analysis is also performed on the SHAP values to identify periodic changes in the contribution of key features.

The beneficial effects of the present disclosure are as follows:

The present disclosure optimizes the candidate indicator set through indicator SHAP data, thereby reducing model parameters and improving the prediction efficiency of the model. Through the improved Keras Sequential model, the integration of data correlations and different training strategies for various types of indicators were achieved, addressing the risk of overfitting during model training and enhancing the prediction accuracy of the model for natural abundance.

Various embodiments in this specification are described progressively, with each embodiment focusing on the differences from other embodiments. For identical or similar parts among the various embodiments, cross-reference may be made accordingly.

Specific examples are used in this text to illustrate the principles and implementation methods of the present disclosure. The description of the above embodiments is only intended to assist in understanding the method and core concepts of the present disclosure. Meanwhile, those skilled in the art may make modifications to the specific embodiments and scope of application based on the concept of the present disclosure. In summary, the content of this specification should not be construed as limiting the present disclosure.

1. A method for predicting natural abundance of nitrogen isotopes during ammonia volatilization based on environmental and soil factors, comprising:
determining a candidate indicator set, and based on the candidate indicator set, performing data collection and data preprocessing in a target research area to obtain a candidate indicator dataset;
collecting natural abundance values of nitrogen isotopes during ammonia volatilization for cropland in the target study area using isotope mass spectrometry analysis to obtain a natural abundance dataset;
constructing a Keras Sequential model, inserting a feature-level self-attention module between an input layer and a first hidden layer, adding a time decay factor before a Dropout layer, replacing an original bias in an activation function of hidden layers with a fixed bias and a variable bias based on fixed and variable quantities in the candidate indicator set, and determining activation of the fixed bias and the variable bias through an indicator function to obtain an improved Keras Sequential model;
constructing a loss function, and iteratively training the improved Keras Sequential model using the candidate indicator dataset and the natural abundance dataset based on the loss function and an early stopping method to obtain an original natural abundance prediction model;
calculating a SHAP value of each candidate indicator in the original natural abundance prediction model using a DeepSHAP interpreter to obtain the candidate indicator SHAP dataset;
filtering the candidate indicator SHAP dataset using a preset SHAP threshold to obtain an updated candidate indicator set;
training the improved Keras Sequential model using the candidate indicator dataset, and the data corresponding to the candidate indicator set in the natural abundance dataset to obtain the indicator-optimized natural abundance prediction model;
calculating an error degree of the indicator-optimized natural abundance prediction model relative to the original natural abundance prediction model using a preset error function to obtain an error ratio;
when the error ratio is less than a preset error threshold, determining the indicator-optimized natural abundance prediction model as a nitrogen isotope natural abundance prediction model; and
performing prediction on the target farmland area using the nitrogen isotope natural abundance prediction model to obtain a prediction result.

2. The method for predicting natural abundance of ammonia volatilization nitrogen isotopes based on environmental and soil factors according to claim 1, wherein the iterative training process of the Keras Sequential model comprises:
converting the candidate indicator dataset into a tensor form to obtain input data;
performing linear transformation, attention weight calculation, weighted fusion, residual connection, and normalization on the input data using the feature-level self-attention module to obtain an attention output;
inputting the attention output into the hidden layer for abstraction processing to obtain a feature tensor;
dynamically retaining and randomly masking the feature tensor using the Dropout layer to obtain a sparsified tensor;
calculating the sparsified tensor successively using the remaining hidden layers and the Dropout layer of the Keras Sequential model to obtain a feature extraction result; and
inputting the feature extraction result into a fully connected layer for mapping of nitrogen isotope abundance values in ammonia volatilization, to obtain an iterative prediction result.

3. The method for predicting natural abundance of nitrogen isotopes in ammonia volatilization based on environmental and soil factors according to claim 1, wherein the activation function is expressed as follows:
f(xᵢ)=ReLU(wᵢxᵢ+b_{fixed}·I(Tₓ_{ᵢ}∈T₀)+bᵥₐᵣ·I(Tₓ_{ᵢ}∈T₁));
wherein f(xᵢ) is an output of the activation function; ReLU(·) is the activation function; wᵢ is a weight parameter; xᵢ is an eigenvalue; b_{fixed} is a fixed bias; bᵥₐᵣ is a variable bias; I(·) is an indicator function; Tₓ_{ᵢ} is an indicator type corresponding to the eigenvalue; T₀ represents a fixed indicator; and T₁ represents a variable indicator.

4. The method for predicting natural abundance of nitrogen isotopes during ammonia volatilization based on environmental and soil factors according to claim 1, wherein the error function is expressed as follows:
$$E = \sum \frac{\vert \text{?} - {y}_{n} \vert}{\vert \text{?} - {y}_{n} \vert} ;$$
wherein E is an error ratio; [symbol] is a prediction result of the original natural abundance prediction model; [symbol] is a prediction result of the indicator-optimized natural abundance prediction model; and yₙ is a measured value of the natural abundance.

5. The method for predicting natural abundance of nitrogen isotopes during ammonia volatilization based on environmental and soil factors according to claim 1, wherein the determined candidate indicator set comprises: air temperature, atmospheric humidity, soil pH, soil ammonium-nitrogen concentration, soil nitrate-nitrogen concentration, soil type, land use type, fertilization type, nitrogen fertilizer application rate, temperature change rate, humidity change rate, ammonium-nitrogen concentration change rate, nitrate-nitrogen concentration change rate, precipitation, wind speed, light intensity, soil moisture, and soil electrical conductivity.

6. The method for predicting natural abundance of nitrogen isotopes during ammonia volatilization based on environmental and soil factors according to claim 1, wherein the SHAP threshold is ranged from 0.1 to 0.2.

7. The prediction method for natural abundance of nitrogen isotopes during ammonia volatilization based on environmental and soil factors according to claim 1, wherein the data preprocessing comprises: outlier removal, linear interpolation, mode imputation, and data normalization.

8. The method for predicting natural abundance of nitrogen isotopes during ammonia volatilization based on environmental and soil factors according to claim 2, wherein the feature-level self-attention module is expressed as follows:
$$Output = LayerNorm ( X + Softmax ( \frac{{XW}_{Q} \cdot {( {XW}_{K} )}^{T}}{\sqrt{d}} ) \cdot ( {XW}_{V} ) \cdot {W}_{O} ) ;$$
wherein Output is an attention output; LayerNorm(·) represents a layer normalization operation; Softmax(·) represents a normalized exponential function; X is an input data matrix; W_{Q}, W_{K}, and W_{V} and are a query matrix, a key matrix, and a value matrix, respectively; W_{O} is an output projection weight matrix; and d is a scaling factor.

9. The method for predicting natural abundance of nitrogen isotopes during ammonia volatilization based on environmental and soil factors according to claim 2, wherein the Dropout layer is expressed as follows:
$$Dropout ( {X}_{t} , t ) = \{ \begin{matrix}\frac{{X}_{t} ⊙ {M}_{t}}{p ( t )} \\ {X}_{t}\end{matrix} ;$$
wherein Dropout(Xₜ, t) is an output of the Dropout layer; Xₜ is an input tensor; Mₜ is a random mask matrix; p(t) is a retention probability; and ⊙ represents an element-wise multiplication operation.

10. The method for predicting natural abundance of nitrogen isotopes during ammonia volatilization based on environmental and soil factors according to claim 5, wherein the collection equipment for the candidate indicator set comprises: a glass electrode pH sensor, a digital ammonia nitrogen and nitrate nitrogen measurement sensor, a resistive soil moisture sensor, and a conductivity sensor.
